(19) United States
(12) Patent Application Publication
(10) Pub. No.: US 2006/0275909 A1
(43) Pub. Date: Dec. 7, 2006

(54) Nutrition formulations and methods of providing nutrition formulations

(76) Inventors: Alan R. Spitzer, Coral Springs, FL (US); Reese H. Clark, Weston, FL (US); Donald H. Chace, Pittsburgh, PA (US)

Correspondence Address: CHOATE, HALL & STEWART LLP, TWO INTERNATIONAL PLACE, BOSTON, MA, 02110, US

(21) Appl. No.: 11/440,311

(22) Filed: May 24, 2006

Related U.S. Application Data
(60) Provisional application No. 60/684,865, filed on May 26, 2005.

(51) Int. Cl. G01N 33/00; A61K 31/714; A61K 31/59; A61K 31/525; A61K 31/51; A61K 31/455; A61K 31/4415; A61K 31/405; A61K 31/4188; A61K 31/4172; A61K 31/375; A61K 31/198; A61K 31/401; A61K 31/122

(52) U.S. Cl. 436/86; 514/52; 514/251; 514/276; 514/167; 514/350; 514/356; 514/393; 514/458; 514/419; 514/400; 514/475; 514/423; 514/561; 514/725; 514/682

(57) ABSTRACT

Nutritional compositions such as total parenteral nutrition (TPN) compositions and processes of preparing same are disclosed. The advantage of the present invention lies in its ability to tailor TPN composition to the needs of each individual patient. Various components of the TPN composition may be increased, lowered, or removed based on the measurement of concentration of components and/or their metabolites in patient's blood.

### RELATED APPLICATIONS

The present application claims priority under 35 U.S.C. § 119(e) to U.S. provisional patent application, U.S. Ser. No. 60/684,865, filed May 26, 2005, which is incorporated herein by reference.

### FIELD OF THE INVENTION

The present invention is directed to nutritional compositions containing amino acids and/or vitamins and processes of their preparation.

### BACKGROUND OF THE INVENTION

Oral supplementation with energy- and protein-rich foods is indicated for patients on modified consistency diets, for those with chronic disease and anorexia, and for those with chronic inflammatory disease or malignancy. In practice, commercial products provide a more reliable and acceptable method of supplementation than table foods.

Total parenteral nutrition (TPN) supplies all of the patient's daily nutritional requirements. TPN is used not only in the hospital for long-term administration but also at home (home TPN), enabling many persons who have lost small-bowel function to lead useful lives.

Severely malnourished patients who are being prepared for surgery, radiation therapy, or chemotherapy for cancer are given TPN before and after treatment to improve and maintain their nutritional status. In major surgery, severe burns, and multiple fractures, especially in the presence of sepsis, TPN reduces subsequent morbidity and mortality, promotes tissue repair, and enhances the immune response. Prolonged coma and anorexia often require TPN after intensive enteral feeding in the early stages. Conditions requiring complete bowel rest (e.g., some stages of Crohn's disease, ulcerative colitis, severe pancreatitis) and pediatric GI disorders (e.g., congenital anomalies, protracted nonspecific diarrhea) often respond well to TPN. Many premature infants who are unable to feed, and critically ill neonates admitted to neonatal intensive care units, also commonly benefit from TPN administration.

TPN requires water (30 to 40 mL/kg/day) and energy (30 to 60 kcal/kg/day), depending on energy expenditure, and amino acids (1 to 3 g/kg/day), depending on the degree of catabolism. Additionally, vitamins and minerals may also present in TPN. The following is a label indicated composition of TROPHAMINE, a well known in the art TPN solution.

| Amino Acids | 6% solution | | 10% solution | |
|---|---|---|---|---|
| Isoleucine USP | 0.49 | g | 0.82 | g |
| Leucine USP | 0.84 | g | 1.4 | g |
| Lysine | 0.49 | g | 0.82 | g |
| (added as Lysine Acetate | 0.69 | g | 1.2 | g) |
| USP | | | | |
| Methionine USP | 0.20 | g | 0.34 | g |
| Phenylalanine USP | 0.29 | g | 0.48 | g |
| Threonine USP | 0.25 | g | 0.42 | g |
| Tryptophan USP | 0.12 | g | 0.20 | g |
| Valine USP | 0.47 | g | 0.78 | g |
| Cysteine | <0.014 | g | <0.016 | g |
| (as Cysteine HCl.H₂O USP | <0.020 | g | <0.024 | g) |
| Histidine USP | 0.29 | g | 0.48 | g |
| Tyrosine | 0.14 | g | 0.24 | g |
| (added as Tyrosine USP | 0.044 | g | 0.044 | g |
| and N-Acetyl-L-Tyrosine | 0.12 | g | 0.24 | g) |
| Alanine USP | 0.32 | g | 0.54 | g |
| Arginine USP | 0.73 | g | 1.2 | g |
| Proline USP | 0.41 | g | 0.68 | g |
| Serine USP | 0.23 | g | 0.38 | g |
| Glycine USP | 0.22 | g | 0.36 | g |
| L-Aspartic Acid | 0.19 | g | 0.32 | g |
| L-Glutamic Acid | 0.30 | g | 0.50 | g |
| Taurine | 0.015 | g | 0.025 | g |
| Sodium Matabisulfite NF | <0.050 | g | <0.050 | g |
| (as an antioxidant) | | | | |

| | | |
|---|---|---|
| Water for Injection USP | qs | qs |

pH adjusted with Glacial Acetic Acid USP
pH: 5.5 (5.0-6.0)

Electrolytes (mEq/liter)

| | | |
|---|---|---|
| Sodium | 5 5 | |
| Acetate (CH₃COO—) | 54.4 | 97 |
| [provided as acetic acid and | | |
| lysine acetate] | | |
| Chloride | <3 | <3 |

Prematurely born infants currently require TPN treatments during their hospitalization. Many problems related to abnormal amino acid metabolism produce abnormal concentrations of ammonia, a result of increased turnover of amino acids for energy production or an indicator of alterations in urea cycle metabolism.

From the newborn screening perspective, time is a critical component in the disease etiology. For most disorders, maternal metabolism insures near normal concentrations of amino acids; after birth, however, deviations from endogenous metabolism are no longer kept in check. With time and other influences such as diet, cell/protein turnover and numerous other factors, both deviations from normal and compensatory mechanisms may occur. Many disorders have been viewed in children who exhibit serious symptoms of disease, in which the abnormal biochemistry is quite evident. In newborn screening, however, since many of these processes have only just begun and no outward medical problems have yet presented, conditions in infants are all too easily affected by small changes in diet, collection time, and other such factors. Therefore, newborn screening will always remain a screening tool and never be 100% accurate. Newer technologies such as tandem mass spectrometry (MS/MS) have impacted newborn screening in a way that has allowed it to more closely approach 100% disease detection through the use of more accurate technologies and multi-analyte approaches.

Phenylketonuria is one amino acid metabolism disorder categorized as a defect in the metabolism of phenylalanine caused by a deficiency of the enzyme phenylalanine hydroxylase. The concentration of phenylalanine in blood is affected by the influx of phenylalanine into the blood stream through dietary absorption, i.v. administration, and protein breakdown. The rate of elimination or turnover of phenylalanine is affected primarily by the enzyme phenylalanine hydroxylase (irreversible enzyme) and secondarily through phenylalanine transaminase (reversible enzyme). The products of phenylalanine that may be important in the assessment of phenylalanine metabolism include tyrosine, phenylpyruvic acid, phenylethalamine, phenylacetate, phenylacetylglutamine, and hydroxyphenylacetate.

Significantly, prematurely born infants receiving TPN treatments may not be able to properly metabolize all TPN components, such as certain amino acids, even if the infants do not have a metabolic disorder. Therefore, prematurely born infants receiving TPN treatments may be at risk of receiving inadequate or excessive amounts of certain amino acids. Currently practiced nutritional adjustment strategies involve changing diet based on detection of a specific metabolic disorder. However, there are no known methods for providing adequate and safe TPN treatments to prematurely born but otherwise healthy infants who may be temporarily unable to metabolize certain components of the TPN solution.

Therefore, it would be advantageous to detect temporary inability to properly metabolize certain TPN components, as well as actual metabolic disorders, as early as possible and adjust TPN composition to avoid administration of an inadequate or excessive dose of any one of the components of the TPN solution, such as a specific amino acid or amino acids. It would also be advantageous to have a relatively simple and reliable process that does not require large quantities of blood samples and which process would allow physician to alter composition of TPN based on observation of concentrations of various analytes in patient's blood.

### SUMMARY OF THE INVENTION

The present invention is directed to nutritional compositions such as total parenteral nutrition (TPN) compositions and processes of preparing same. The advantage of the present invention lies in its ability to tailor TPN composition to the needs of each individual patient. Various components of the TPN composition may be increased, lowered, or removed based on the measurement of concentration of components and/or their metabolites in patient's blood.

Another advantage of the present invention lies in recognition of existence of a problem in administration of standard TPN treatments to prematurely born infants who do not have any specific metabolic disorders but who are unable to properly metabolize some of the components of the TPN solution due to their developmental stage. The present invention solves this problem by providing a method for monitoring metabolism of TPN solutions by prematurely born infants and accordingly adjusting composition of TPN solutions.

In one embodiment, the invention is directed to a nutritional composition comprising supplemental amino acids, prepared by a process comprising: a) determining patient's concentration of at least one blood amino acid indicator; b) observing if concentration of the at least one blood amino acid indicator determined in step (a) is above or below normal concentration; and c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator. Step (a) may be performed by collecting patient's blood on a filter paper and analyzing patient's blood on the filter paper with a tandem mass spectrometer.

In another embodiment, the invention is directed to a process of preparation of nutritional composition comprising amino acids, the process comprising: a) determining patient's concentration of at least one blood amino acid indicator; b) observing if concentration of the at least one blood amino acid indicator determined in step (a) is above or below normal concentration; and c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator. Step (a) may be performed by collecting patient's blood on a filter paper and analyzing patient's blood on the filter paper with a tandem mass spectrometer.

The patient's blood may be tested for and the TPN solutions of the invention may also be adjusted for other components besides amino acids, such as vitamins.

In another embodiment, the nutritional composition contains proteins and measurements of concentrations of blood amino acid indicators are used to correspondingly adjust protein diet. For example, above normal concentrations of blood amino acid indicators would require reduced levels of proteins in the nutritional composition.

### DETAILED DESCRIPTION OF THE INVENTION

A term “nutritional composition” is meant to encompass a composition for administration to a patient, which serves the purpose of providing nutrition or providing supplemental nutrition to a patient. One example of a nutritional composition is a total parenteral nutrition (TPN) solution containing amino acids and/or vitamins.

A term “patient” is meant to encompass an animal, such as human, who may benefit from nutritional compositions of the invention, and who may or may not suffer from an ailment. In one preferred embodiment, the patient is a human infant. In another preferred embodiment, the patient is a prematurely born human infant. In yet another preferred embodiment, the patient is a prematurely born human infant who does not have a metabolic disorder.

A term “amino acid” is meant to encompass any organic acid containing one or more amino substituents. It is meant to encompass both α-amino and β-amino derivatives of aliphatic carboxylic acids. It is also meant to encompass so-called “essential amino acids” such as isoleucine, leucine, valine, threonine, methionine, tryptophan, phenylalanine, and lysine; so-called “semi-essential amino acids” such as histidine, tyrosine, cysteine, and taurine; and “non-essential amino acids” such as glycine, alanine, praline, serine, arginine, aspartic acid, and glutamine. The amino acids of the invention may be present in the composition in the form of pharmaceutically acceptable salts. For example, cysteine may be present as an aqueous hydrochloride salt solution. The amino acids of the invention may also be present in a modified form. For example, lysine may be present as lysine and/or as lysine acetate. Similarly, tyrosine may be present as a mixture of tyrosine and N-acetyl-L-tyrosine.

The term “pharmaceutically acceptable salt” refers to salts prepared from pharmaceutically acceptable non-toxic acids or bases including inorganic acids and bases and organic acids and bases. When the compounds used in the present invention are basic, salts may be prepared from pharmaceutically acceptable non-toxic acids including inorganic and organic acids. Suitable pharmaceutically acceptable acid addition salts for the compounds used in the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds used in the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

A term “supplemental amino acid” is meant to encompass amino acid that is present in nutritional composition.

A term “blood amino acid indicator” is meant to encompass amino acid that is present in patient's blood. This term also encompasses metabolite or metabolites of any one specific amino acid as well as combinations of amino acid and its metabolite(s).

The term “metabolite” refers to a product of metabolism and is meant to encompass metabolites of metabolites.

A term “vitamin” is meant to encompass any of various organic substances that are essential in minute quantities to the nutrition, act as coenzymes and precursors of coenzymes in the regulation of metabolic processes but do not provide energy or serve as building units, and are present in natural foodstuffs or are sometimes produced within the body. Examples of vitamins are ascorbic acid, vitamin A, vitamin D, thiamine, riboflavin, pyridoxine, niacinamide, dexapanthenol, vitamin E, biotin, folic acid, vitamin B12, and vitamin K.

A term “blood vitamin indicator” is meant to encompass a vitamin present in patient's blood. This term also encompasses metabolite or metabolites of any one specific vitamin as well as combinations of vitamin and its metabolite(s).

A term “supplemental vitamin” is meant to encompass a vitamin present in nutritional composition.

A term “patient's concentration” is meant to encompass concentration of an amino acid or vitamin in patient's blood.

A term “normal concentration” is meant to encompass a concentration of amino acid(s), of vitamin(s), or of other substances that is observed in blood of a healthy subject.

A term “above normal concentration” is meant to encompass a concentration in patient's blood of amino acid(s) or vitamin(s) that is higher than concentration of respective amino acid(s) or vitamin(s) in healthy subject with physiological parameters that are similar to those of the patient.

A term “below normal concentration” is meant to encompass a concentration in patient's blood of amino acid(s) or vitamin(s) that is lower than concentration of respective amino acid(s) or vitamin(s) in healthy subject with physiological parameters that are similar to those of the patient.

A term “corresponding” as used in the present claims has meaning of being of the same identity but it also includes non-identical molecules such as an amino acid and its metabolite(s) and mixtures thereof, as well as vitamin and its metabolite(s) and mixtures thereof. Thus, a blood amino acid indicator may be a metabolite of such amino acid as phenylalanine, having phenylalanine as a corresponding supplemental amino acid.

A term “inverse correlation” is meant to encompass a relationship wherein an increase in one value corresponds to a decrease in another value and vice versa. For example, according to the present invention an observation of an above normal concentration of phenylalanine in patient's blood would require providing a nutritional composition with a lowered concentration of phenylalanine as compared to a standard concentration of phenylalanine in parenteral solution. Similarly, according to the present invention an observation of a below normal concentration of phenylalanine in patient's blood would require providing a nutritional composition with an increased concentration of phenylalanine as compared to a standard concentration of phenylalanine in parenteral solution. The degree of increase or decrease in concentration of an amino acid or a vitamin in nutritional composition is approximately proportional to corresponding deviation from normal in concentration of blood amino acid indicator or blood vitamin indicator.

A term “filter paper” is meant to encompass specimen collection paper that is well known in the art. Some known examples are Schleicher & Schuell's “Grade 903” filter papers and Whatman's “BFC 180” filter papers. Methods of collection of blood samples on filter papers are well known in the art.

A term “tandem mass spectrometer” is meant to encompass a well known in the art instrument consisting of two mass spectrometers in series connected by a chamber known as a collision cell. The sample to be examined is essentially sorted and weighed in the first mass spectrometer, then broken into pieces in the collision cell, and a piece or pieces sorted and weighed in the second mass spectrometer. Tandem mass spectrometry is used in newborn screening to detect molecules such as amino acids and fatty acids.

In one embodiment, the invention is directed to a nutritional composition comprising supplemental amino acids, prepared by a process comprising: a) determining patient's concentration of at least one blood amino acid indicator; b) observing if concentration of the at least one blood amino acid indicator determined in step (a) is above or below normal concentration; and c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator. Step (a) may be performed by collecting patient's blood on a filter paper and analyzing patient's blood on the filter paper with a tandem mass spectrometer.

In another embodiment, the invention is directed to a nutritional composition comprising supplemental amino acids, prepared by a process comprising: a) collecting patient's blood on a filter paper; b) analyzing patient's blood on the filter paper of step (a) with a tandem mass spectrometer; c) observing from step (b) concentration of an at least one blood amino acid indicator; d) determining if the concentration of the at least one blood amino acid indicator of step (c) is above or below normal concentration; and e) providing nutritional composition comprising supplemental amino acids, wherein an at least one supplemental amino acid corresponds to the at least one blood amino acid indicator of step (d) and wherein concentration of the at least one supplemental amino acid is in inverse correlation with the concentration of the at least one blood amino acid indicator.

One example of above two embodiments would be a parenteral nutrition solution having half the standard concentration of phenylalanine when an observation is made of an increased concentration of phenylalanine in the blood of the patient.

Another example of above two embodiments would be a parenteral nutrition solution having double the standard concentration of isoleucine when an observation is made of a decreased concentration of isoleucine in the blood of the patient.

In another embodiment, the invention is directed to a nutritional composition comprising supplemental amino acids, prepared by a process comprising: a) determining patient's concentration of at least one blood amino acid indicator; b) observing if concentration of the at least one blood amino acid indicator determined in step (a) is above normal concentration; and c) providing nutritional composition comprising supplemental amino acids, wherein an at least one supplemental amino acid that corresponds to the at least one blood amino acid indicator of step (b) is absent. Step (a) may be performed by collecting patient's blood on a filter paper and analyzing patient's blood on the filter paper with a tandem mass spectrometer.

In another embodiment, the invention is directed to a nutritional composition comprising supplemental amino acids, prepared by a process comprising: a) collecting patient's blood on a filter paper; b) analyzing patient's blood on the filter paper of step (a) with a tandem mass spectrometer; c) observing from step (b) concentration of an at least one blood amino acid indicator; d) determining if the concentration of the at least one blood amino acid indicator of step (c) is above normal concentration; and e) providing nutritional composition comprising supplemental amino acids, wherein an at least one supplemental amino acid that corresponds to the at least one blood amino acid indicator of step (d) is absent.

One example of above two embodiments would be a parenteral nutrition solution having no phenylalanine when an observation is made of an increased concentration of phenylalanine and/or its metabolite(s) in the blood of the patient.

In another embodiment, the invention is directed to a process of preparation of nutritional composition comprising amino acids, the process comprising: a) determining patient's concentration of at least one blood amino acid indicator; b) observing if concentration of the at least one blood amino acid indicator determined in step (a) is above or below normal concentration; and c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator. Step (a) may be performed by collecting patient's blood on a filter paper and analyzing patient's blood on the filter paper with a tandem mass spectrometer.

In another embodiment, the invention is directed to a process of preparation of nutritional composition comprising supplemental amino acids, the process comprising: a) collecting patient's blood on a filter paper; b) analyzing patient's blood on the filter paper of step (a) with a tandem mass spectrometer; c) observing from step (b) concentration of an at least one blood amino acid indicator; d) determining if the concentration of the at least one blood amino acid indicator of step (c) is above or below normal concentration; and e) providing nutritional composition comprising supplemental amino acids, wherein an at least one supplemental amino acid corresponds to the at least one blood amino acid indicator of step (d) and wherein concentration of the at least one supplemental amino acid is in inverse correlation with the concentration of the at least one blood amino acid indicator.

In another embodiment, the invention is directed to a process of preparation of nutritional composition comprising amino acids, the process comprising: a) determining patient's concentration of at least one blood amino acid indicator; b) observing if concentration of the at least one blood amino acid indicator determined in step (a) is above normal concentration; and c) providing nutritional composition comprising supplemental amino acids, wherein an at least one supplemental amino acid that corresponds to the at least one blood amino acid indicator of step (b) is absent. Step (a) may be performed by collecting patient's blood on a filter paper and analyzing patient's blood on the filter paper with a tandem mass spectrometer.

In another embodiment, the invention is directed to a process of preparation of nutritional composition comprising supplemental amino acids, the process comprising: a) collecting patient's blood on a filter paper; b) analyzing patient's blood on the filter paper of step (a) with a tandem mass spectrometer; c) observing from step (b) concentration of an at least one blood amino acid indicator; d) determining if the concentration of the at least one blood amino acid indicator of step (c) is above normal concentration; and e) providing nutritional composition comprising supplemental amino acids, wherein an at least one supplemental amino acid that corresponds to the at least one blood amino acid indicator of step (d) is absent.

The present invention is also directed to a nutritional composition comprising supplemental vitamins, prepared by a process comprising: a) determining patient's concentration of at least one blood vitamin indicator; b) observing if concentration of the at least one blood vitamin indicator determined in step (a) is above or below normal concentration; and c) providing nutritional composition comprising supplemental vitamins, wherein an at least one supplemental vitamin corresponds to the at least one blood vitamin indicator of step (b) and wherein concentration of the at least one supplemental vitamin is in inverse correlation with the concentration of the at least one blood vitamin indicator. Step (a) may be performed by collecting patient's blood on a filter paper and analyzing patient's blood on the filter paper with a tandem mass spectrometer.

In another embodiment, the invention is directed to a nutritional composition comprising supplemental vitamins, prepared by a process comprising: a) collecting patient's blood on a filter paper; b) analyzing patient's blood on the filter paper of step (a) with a tandem mass spectrometer; c) observing from step (b) concentration of an at least one blood vitamin indicator; d) determining if the concentration of the at least one blood vitamin indicator of step (c) is above or below normal concentration; and e) providing nutritional composition comprising supplemental vitamins, wherein an at least one supplemental vitamin corresponds to the at least one blood vitamin indicator of step (d) and wherein concentration of the at least one supplemental vitamin is in inverse correlation with the concentration of the at least one blood vitamin indicator.

One example of above two embodiments would be a parenteral nutrition solution having half the standard concentration of vitamin K when an observation is made of an increased concentration of vitamin K in the blood of the patient.

Another example of above two embodiments would be a parenteral nutrition solution having double the standard concentration of ascorbic acid when an observation is made of a decreased concentration of ascorbic acid or its metabolites in the blood of the patient.

In another embodiment, the invention is directed to a nutritional composition comprising supplemental vitamins, prepared by a process comprising: a) determining patient's concentration of at least one blood vitamin indicator; b) observing if concentration of the at least one blood vitamin indicator determined in step (a) is above normal concentration; and c) providing nutritional composition comprising supplemental vitamins, wherein an at least one supplemental vitamin that corresponds to the at least one blood vitamin indicator of step (b) is absent. Step (a) may be performed by collecting patient's blood on a filter paper and analyzing patient's blood on the filter paper with a tandem mass spectrometer.

In another embodiment, the invention is directed to a nutritional composition comprising supplemental vitamins, prepared by a process comprising: a) collecting patient's blood on a filter paper; b) analyzing patient's blood on the filter paper of step (a) with a tandem mass spectrometer; c) observing from step (b) concentration of an at least one blood vitamin indicator; d) determining if the concentration of the at least one blood vitamin indicator of step (c) is above normal concentration; and e) providing nutritional composition comprising supplemental vitamins, wherein an at least one supplemental vitamin that corresponds to the at least one blood vitamin indicator of step (d) is absent.

One example of above two embodiments would be a parenteral nutrition solution having no niacinamide when an observation is made of an increased concentration of niacinamide or its metabolites in the blood of the patient.

In another embodiment, the invention is directed to a process of preparation of nutritional composition comprising vitamins, the process comprising: a) determining patient's concentration of at least one blood vitamin indicator; b) observing if concentration of the at least one blood vitamin indicator determined in step (a) is above or below normal concentration; and c) providing nutritional composition comprising supplemental vitamins, wherein an at least one supplemental vitamin corresponds to the at least one blood vitamin indicator of step (b) and wherein concentration of the at least one supplemental vitamin is in inverse correlation with the concentration of the at least one blood vitamin indicator. Step (a) may be performed by collecting patient's blood on a filter paper and analyzing patient's blood on the filter paper with a tandem mass spectrometer.

In another embodiment, the invention is directed to a process of preparation of nutritional composition comprising supplemental vitamins, the process comprising: a) collecting patient's blood on a filter paper; b) analyzing patient's blood on the filter paper of step (a) with a tandem mass spectrometer; c) observing from step (b) concentration of an at least one blood vitamin indicator; d) determining if the concentration of the at least one blood vitamin indicator of step (c) is above or below normal concentration; and e) providing nutritional composition comprising supplemental vitamins, wherein an at least one supplemental vitamin corresponds to the at least one blood vitamin indicator of step (d) and wherein concentration of the at least one supplemental vitamin is in inverse correlation with the concentration of the at least one blood vitamin indicator.

In another embodiment, the invention is directed to a process of preparation of nutritional composition comprising vitamins, the process comprising: a) determining patient's concentration of at least one blood vitamin indicator; b) observing if concentration of the at least one blood vitamin indicator determined in step (a) is above normal concentration; and c) providing nutritional composition comprising supplemental vitamins, wherein an at least one supplemental vitamin that corresponds to the at least one blood vitamin indicator of step (b) is absent. Step (a) may be performed by collecting patient's blood on a filter paper and analyzing patient's blood on the filter paper with a tandem mass spectrometer.

In another embodiment, the invention is directed to a process of preparation of nutritional composition comprising supplemental vitamins, the process comprising: a) collecting patient's blood on a filter paper; b) analyzing patient's blood on the filter paper of step (a) with a tandem mass spectrometer; c) observing from step (b) concentration of an at least one blood vitamin indicator; d) determining if the concentration of the at least one blood vitamin indicator of step (c) is above normal concentration; and e) providing nutritional composition comprising supplemental vitamins, wherein an at least one supplemental vitamin that corresponds to the at least one blood vitamin indicator of step (d) is absent.

In one preferred embodiment patient's concentration of blood amino acid indicators and/or blood vitamin indicators is measured daily with corresponding TPN composition adjustment. In another preferred embodiment patient's concentration of blood amino acid indicators and/or blood vitamin indicators is measured approximately every 12 hours. In some cases it may be necessary to measure patient's concentration of blood amino acid indicators and/or blood vitamin indicators at more frequent intervals. Since prematurely born infants without metabolic disorders with time become more competent in their ability to metabolize various components of the TPN solution, continuous monitoring of the composition of their blood allows for appropriate adjustments to be made to the TPN solution composition.

The formulations of the present invention include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association amino acids and/or vitamins or their pharmaceutically acceptable salts or solvates thereof (“active ingredients”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

Formulations for parenteral administration are preferred and include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

It should be understood that in addition to the ingredients particularly mentioned above, the formulations of this invention may include other agents conventional in the art having regard to the type of formulation in question, for example those suitable for oral administration may include flavoring agents.

### EXAMPLES

### Example 1

### High Isoleucine, High Leucine, Low Phenylalanine Amino Acid Parenteral Nutrition Solution

Following is an example formulation of an amino acid parenteral nutrition solution adjusted from standard TROPHAMINE solution by having increased concentration of isoleucine and leucine while having reduced concentration of phenylalanine.

| | Adjusted 6% | | Adjusted 10% | |
|---|---|---|---|---|
| Amino Acids | solution | | solution | |
| Isoleucine USP | 0.98 | g | 1.64 | g |
| Leucine USP | 1.68 | g | 2.8 | g |
| Lysine | 0.49 | g | 0.82 | g |
| (added as Lysine Acetate | 0.69 | g | 1.2 | g) |
| USP | | | | |
| Methionine USP | 0.20 | g | 0.34 | g |
| Phenylalanine USP | 0.15 | g | 0.25 | g |
| Threonine USP | 0.25 | g | 0.42 | g |
| Tryptophan USP | 0.12 | g | 0.20 | g |
| Valine USP | 0.47 | g | 0.78 | g |
| Cysteine | <0.014 | g | <0.016 | g |
| (as Cysteine HCl.H₂O USP | <0.020 | g | <0.024 | g) |
| Histidine USP | 0.29 | g | 0.48 | g |
| Tyrosine | 0.14 | g | 0.24 | g |
| (added as Tyrosine USP | 0.044 | g | 0.044 | g |
| and N-Acetyl-L-Tyrosine | 0.12 | g | 0.24 | g) |
| Alanine USP | 0.32 | g | 0.54 | g |
| Arginine USP | 0.73 | g | 1.2 | g |
| Proline USP | 0.41 | g | 0.68 | g |
| Serine USP | 0.23 | g | 0.38 | g |
| Glycine USP | 0.22 | g | 0.36 | g |
| L-Aspartic Acid | 0.19 | g | 0.32 | g |
| L-Glutamic Acid | 0.30 | g | 0.50 | g |
| Taurine | 0.015 | g | 0.025 | g |
| Sodium Matabisulfite NF | <0.050 | g | <0.050 | g |
| (as an antioxidant) | | | | |

| | | |
|---|---|---|
| Water for Injection USP | qs | qs |

pH adjusted with Glacial Acetic Acid USP
pH: 5.5 (5.0-6.0)

Electrolytes (mEq/liter)

| | | |
|---|---|---|
| Sodium | 5 5 | |
| Acetate (CH₃COO—) | 54.4 | 97 |
| [provided as acetic acid and | | |
| lysine acetate] | | |
| Chloride | <3 | <3 |

### Example 2

### Reduced Phenylalanine Concentration Amino Acid Parenteral Nutrition Solution

Following is an example formulation of an amino acid parenteral nutrition solution adjusted from standard TROPHAMINE solution by having reduced concentration of phenylalanine.

| | Adjusted 6% | | Adjusted 10% | |
|---|---|---|---|---|
| Amino Acids | solution | | solution | |
| Isoleucine USP | 0.49 | g | 0.82 | g |
| Leucine USP | 0.84 | g | 1.4 | g |
| Lysine | 0.49 | g | 0.82 | g |
| (added as Lysine Acetate | 0.69 | g | 1.2 | g) |
| USP | | | | |
| Methionine USP | 0.20 | g | 0.34 | g |
| Phenylalanine USP | 0.10 | g | 0.16 | g |
| Threonine USP | 0.25 | g | 0.42 | g |
| Tryptophan USP | 0.12 | g | 0.20 | g |
| Valine USP | 0.47 | g | 0.78 | g |
| Cysteine | <0.014 | g | <0.016 | g |
| (as Cysteine HCl.H₂O USP | <0.020 | g | <0.024 | g) |
| Histidine USP | 0.29 | g | 0.48 | g |
| Tyrosine | 0.14 | g | 0.24 | g |
| (added as Tyrosine USP | 0.044 | g | 0.044 | g |
| and N-Acetyl-L-Tyrosine | 0.12 | g | 0.24 | g) |
| Alanine USP | 0.32 | g | 0.54 | g |
| Arginine USP | 0.73 | g | 1.2 | g |
| Proline USP | 0.41 | g | 0.68 | g |
| Serine USP | 0.23 | g | 0.38 | g |
| Glycine USP | 0.22 | g | 0.36 | g |
| L-Aspartic Acid | 0.19 | g | 0.32 | g |
| L-Glutamic Acid | 0.30 | g | 0.50 | g |
| Taurine | 0.015 | g | 0.025 | g |
| Sodium Matabisulfite NF | <0.050 | g | <0.050 | g |
| (as an antioxidant) | | | | |

| | | |
|---|---|---|
| Water for Injection USP | qs | qs |

pH adjusted with Glacial Acetic Acid USP
pH: 5.5 (5.0-6.0)

Electrolytes (mEq/liter)

| | | |
|---|---|---|
| Sodium | 5 5 | |
| Acetate (CH₃COO—) | 54.4 | 97 |
| [provided as acetic acid and | | |
| lysine acetate] | | |
| Chloride | <3 | <3 |

### Example 3

### Phenylalanine Free Amino Acid Parenteral Nutrition Solution

Following is an example formulation of an amino acid parenteral nutrition solution adjusted from standard TROPHAMINE solution by having no phenylalanine.

| | Adjusted 6% | | Adjusted 10% | |
|---|---|---|---|---|
| Amino Acids | solution | | solution | |
| Isoleucine USP | 0.49 | g | 0.82 | g |
| Leucine USP | 0.84 | g | 1.4 | g |
| Lysine | 0.49 | g | 0.82 | g |
| (added as Lysine Acetate | 0.69 | g | 1.2 | g) |
| USP | | | | |
| Methionine USP | 0.20 | g | 0.34 | g |
| Threonine USP | 0.25 | g | 0.42 | g |
| Tryptophan USP | 0.12 | g | 0.20 | g |
| Valine USP | 0.47 | g | 0.78 | g |
| Cysteine | <0.014 | g | <0.016 | g |
| (as Cysteine HCl.H₂O USP | <0.020 | g | <0.024 | g) |
| Histidine USP | 0.29 | g | 0.48 | g |
| Tyrosine | 0.14 | g | 0.24 | g |
| (added as Tyrosine USP | 0.044 | g | 0.044 | g |
| and N-Acetyl-L-Tyrosine | 0.12 | g | 0.24 | g) |
| Alanine USP | 0.32 | g | 0.54 | g |
| Arginine USP | 0.73 | g | 1.2 | g |
| Proline USP | 0.41 | g | 0.68 | g |
| Serine USP | 0.23 | g | 0.38 | g |
| Glycine USP | 0.22 | g | 0.36 | g |
| L-Aspartic Acid | 0.19 | g | 0.32 | g |
| L-Glutamic Acid | 0.30 | g | 0.50 | g |
| Taurine | 0.015 | g | 0.025 | g |
| Sodium Matabisulfite NF | <0.050 | g | <0.050 | g |
| (as an antioxidant) | | | | |

| | | |
|---|---|---|
| Water for injection USP | qs | qs |

pH adjusted with Glacial Acetic Acid USP
pH: 5.5 (5.0-6.0)

Electrolytes (mEq/liter)

| | | |
|---|---|---|
| Sodium | 5 5 | |
| Acetate (CH₃COO—) | 54.4 | 97 |
| [provided as acetic acid and | | |
| lysine acetate] | | |
| Chloride | <3 | <3 |

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

What is claimed is:

1. A nutritional composition comprising supplemental amino acids, prepared by a process comprising:
a) determining patient's concentration of at least one blood amino acid indicator;
b) observing if concentration of the at least one blood amino acid indicator determined in step (a) is above or below normal concentration; and
c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator.

2. A nutritional composition comprising supplemental amino acids, prepared by a process comprising:
a) collecting patient's blood on a filter paper;
b) analyzing patient's blood on the filter paper of step (a) with a tandem mass spectrometer;
c) observing from step (b) concentration of an at least one blood amino acid indicator;
d) determining if the concentration of the at least one blood amino acid indicator of step (c) is above or below normal concentration; and
e) providing nutritional composition comprising supplemental amino acids, wherein an at least one supplemental amino acid corresponds to the at least one blood amino acid indicator of step (d) and wherein concentration of the at least one supplemental amino acid is in inverse correlation with the concentration of the at least one blood amino acid indicator.

3. A nutritional composition comprising supplemental amino acids, prepared by a process comprising:
a) determining patient's concentration of at least one blood amino acid indicator;
b) observing if concentration of the at least one blood amino acid indicator determined in step (a) is above normal concentration; and
c) providing nutritional composition comprising supplemental amino acids, wherein an at least one supplemental amino acid that corresponds to the at least one blood amino acid indicator of step (b) is absent.

4. A nutritional composition comprising supplemental amino acids, prepared by a process comprising:
a) collecting patient's blood on a filter paper;
b) analyzing patient's blood on the filter paper of step (a) with a tandem mass spectrometer;
c) observing from step (b) concentration of an at least one blood amino acid indicator;
d) determining if the concentration of the at least one blood amino acid indicator of step (c) is above normal concentration; and
e) providing nutritional composition comprising supplemental amino acids, wherein an at least one supplemental amino acid that corresponds to the at least one blood amino acid indicator of step (d) is absent.

5. The nutritional composition of any of claims 1 through 4, wherein the patient is an infant.

6. The nutritional composition of any of claims 1 through 4, wherein the at least one supplemental amino acid is selected from the group consisting of alanine, arginine, aspargine, aspartic acid, cysteine, glutamic acid, glutamine, glycine, histidine, isoleucine, leucine, lysine, methionine, phenylalanine, praline, serine, taurine, threonine, tryptophan, tyrosine, and valine.

7. A process of preparation of nutritional composition comprising amino acids, the process comprising:
a) determining patient's concentration of at least one blood amino acid indicator;
b) observing if concentration of the at least one blood amino acid indicator determined in step (a) is above or below normal concentration; and
c) providing nutritional composition comprising supplemental amino acids, wherein an at least one supplemental amino acid corresponds to the at least one blood amino acid indicator of step (b) and wherein concentration of the at least one supplemental amino acid is in inverse correlation with the concentration of the at least one blood amino acid indicator.

8. A process of preparation of nutritional composition comprising supplemental amino acids, the process comprising:
a) collecting patient's blood on a filter paper;
b) analyzing patient's blood on the filter paper of step (a) with a tandem mass spectrometer;
c) observing from step (b) concentration of an at least one blood amino acid indicator;
d) determining if the concentration of the at least one blood amino acid indicator of step (c) is above or below normal concentration; and
e) providing nutritional composition comprising supplemental amino acids, wherein an at least one supplemental amino acid corresponds to the at least one blood amino acid indicator of step (d) and wherein concentration of the at least one supplemental amino acid is in inverse correlation with the concentration of the at least one blood amino acid indicator.

9. A process of preparation of nutritional composition comprising amino acids, the process comprising:
a) determining patient's concentration of at least one blood amino acid indicator;
b) observing if concentration of the at least one blood amino acid indicator determined in step (a) is above normal concentration; and
c) providing nutritional composition comprising supplemental amino acids, wherein an at least one amino acid that corresponds to the at least one blood amino acid indicator of step (b) is absent.

10. A process of preparation of nutritional composition comprising supplemental amino acids, the process comprising:
a) collecting patient's blood on a filter paper;
b) analyzing patient's blood on the filter paper of step (a) with a tandem mass spectrometer;
c) observing from step (b) concentration of an at least one blood amino acid indicator;
d) determining if the concentration of the at least one blood amino acid indicator of step (c) is above normal concentration; and
e) providing nutritional composition comprising supplemental amino acids, wherein an at least one supplemental amino acid that corresponds to the at least one blood amino acid indicator of step (d) is absent.

11. The process of any of claims 7 through 10, wherein the patient is an infant.

12. The process of any of claims 7 through 10, wherein the at least one supplemental amino acid is selected from the group consisting of alanine, arginine, aspargine, aspartic acid, cysteine, glutamic acid, glutamine, glycine, histidine, isoleucine, leucine, lysine, methionine, phenylalanine, praline, serine, taurine, threonine, tryptophan, tyrosine, and valine.

13. A nutritional composition comprising supplemental vitamins, prepared by a process comprising:
a) determining patient's concentration of at least one blood vitamin indicator;
b) observing if concentration of the at least one blood vitamin indicator determined in step (a) is above or below normal concentration; and
c) providing nutritional composition comprising supplemental vitamins, wherein an at least one supplemental vitamin corresponds to the at least one blood vitamin indicator of step (b) and wherein concentration of the at least one supplemental vitamin is in inverse correlation with the concentration of the at least one blood vitamin indicator.

14. A nutritional composition comprising supplemental vitamins, prepared by a process comprising:
a) collecting patient's blood on a filter paper;
b) analyzing patient's blood on the filter paper of step (a) with a tandem mass spectrometer;
c) observing from step (b) concentration of an at least one blood vitamin indicator;
d) determining if the concentration of the at least one blood vitamin indicator of step (c) is above or below normal concentration; and
e) providing nutritional composition comprising supplemental vitamins, wherein an at least one supplemental vitamin corresponds to the at least one blood vitamin indicator of step (d) and wherein concentration of the at least one supplemental vitamin is in inverse correlation with the concentration of the at least one blood vitamin indicator.

15. A nutritional composition comprising supplemental vitamins, prepared by a process comprising:
a) determining patient's concentration of at least one blood vitamin indicator;
b) observing if concentration of the at least one blood vitamin indicator determined in step (a) is above normal concentration; and
c) providing nutritional composition comprising supplemental vitamins, wherein an at least one supplemental vitamin that corresponds to the at least one blood vitamin indicator of step (b) is absent.

16. A nutritional composition comprising supplemental vitamins, prepared by a process comprising:
a) collecting patient's blood on a filter paper;
b) analyzing patient's blood on the filter paper of step (a) with a tandem mass spectrometer;
c) observing from step (b) concentration of an at least one blood vitamin indicator;
d) determining if the concentration of the at least one blood vitamin indicator of step (c) is above normal concentration; and
e) providing nutritional composition comprising supplemental vitamins, wherein an at least one supplemental vitamin that corresponds to the at least one blood vitamin indicator of step (d) is absent.

17. The nutritional composition of any of claims 13 through 16, wherein the patient is an infant.

18. The nutritional composition of any of claims 13 through 16, wherein the at least one supplemental vitamin is selected from the group consisting of ascorbic acid, vitamin A, vitamin D, thiamine, riboflavin, pyridoxine, niacinamide, dexapanthenol, vitamin E, biotin, folic acid, vitamin B 12, and vitamin K.

19. A process of preparation of nutritional composition comprising vitamins, the process comprising:
a) determining patient's concentration of at least one blood vitamin indicator;
b) observing if concentration of the at least one blood vitamin indicator determined in step (a) is above or below normal concentration; and
c) providing nutritional composition comprising supplemental vitamins, wherein an at least one supplemental vitamin corresponds to the at least one blood vitamin indicator of step (b) and wherein concentration of the at least one supplemental vitamin is in inverse correlation with the concentration of the at least one blood vitamin indicator.

20. A process of preparation of nutritional composition comprising supplemental vitamins, the process comprising:
a) collecting patient's blood on a filter paper;
b) analyzing patient's blood on the filter paper of step (a) with a tandem mass spectrometer;
c) observing from step (b) concentration of an at least one blood vitamin indicator;
d) determining if the concentration of the at least one blood vitamin indicator of step (c) is above or below normal concentration; and
e) providing nutritional composition comprising supplemental vitamins, wherein an at least one supplemental vitamin corresponds to the at least one blood vitamin indicator of step (d) and wherein concentration of the at least one supplemental vitamin is in inverse correlation with the concentration of the at least one blood vitamin indicator.

21. A process of preparation of nutritional composition comprising vitamins, the process comprising:
a) determining patient's concentration of at least one blood vitamin indicator;
b) observing if concentration of the at least one blood vitamin indicator determined in step (a) is above normal concentration; and
c) providing nutritional composition comprising supplemental vitamins, wherein an at least one supplemental vitamin that corresponds to the at least one blood vitamin indicator of step (b) is absent.

22. A process of preparation of nutritional composition comprising supplemental vitamins, the process comprising:
a) collecting patient's blood on a filter paper;
b) analyzing patient's blood on the filter paper of step (a) with a tandem mass spectrometer;
c) observing from step (b) concentration of an at least one blood vitamin indicator;
d) determining if the concentration of the at least one blood vitamin indicator of step (c) is above normal concentration; and
e) providing nutritional composition comprising supplemental vitamins, wherein an at least one supplemental vitamin that corresponds to the at least one blood vitamin indicator of step (d) is absent.

23. The process of any of claims 19 through 22, wherein the patient is an infant.

24. The process of any of claims 19 through 22, wherein the at least one supplemental vitamin is selected from the group consisting of ascorbic acid, vitamin A, vitamin D, thiamine, riboflavin, pyridoxine, niacinamide, dexapanthenol, vitamin E, biotin, folic acid, vitamin B12, and vitamin K.
